(19) United States
(12) Patent Application Publication
(10) Pub. No.: US 2008/0125841 A1
(43) Pub. Date: May 29, 2008

(54) Device for providing instant cooling, particularly for treating parts of the human body or items in general

(75) Inventors: Carlo Ferri, Alzano Lombardo (IT); Adriano Ferrari, Trescore Balneario (IT)

Correspondence Address: MODIANO & ASSOCIATI, Via Meravigli, 16, MILANO, 20123, omitted

(73) Assignee: FERRARI TRADING S.R.L.

(21) Appl. No.: 11/987,142

(22) Filed: Nov. 28, 2007

(30) Foreign Application Priority Data
Nov. 27, 2006 (IT) MI2006A002278

(51) Int. Cl. A61F 7/00

(52) U.S. Cl. 607/107

(57) ABSTRACT

A device for providing instant cooling, particularly for treating parts of the human body or items in general, comprising a diffuser which is adapted to be charged with a gas, the diffuser being provided with a plurality of holes for releasing the gas, the diffuser being accommodated in a container which contains a thermally insulating material which is adapted to absorb the low temperature generated by the gas, the container being sealed around the diffuser.

The present invention relates to a device for providing instant cooling, particularly for treating parts of the human body or items in general. More particularly, the invention relates to a device which is adapted to generate cold or heat instantly, so as to reduce or increase the temperature of parts of the human body for therapeutic purposes or to reduce or increase the temperature of items, such as helmets or bottle-carrying baskets.

### BACKGROUND OF THE INVENTION

As is known, for example after a specific surgical procedure, it is necessary to keep the affected part for a period of time at a temperature which is lower than the rest of the body in order to avoid the onset of acute inflammatory conditions. For this purpose, special plastic containers are normally used which contain chemical components which, when mixed together at the time of use, react and cause a drop in temperature. Such containers, placed in contact with the affected part, cool it.

Although these containers are widely used, their drawback is the fact that they have a preset duration, and once the chemical reaction has ended they remain inert, no longer generating cold, and in any case they do not offer temperature control. Substantially, this means that the containers go from being extremely cold when the chemical reaction is triggered, with discomfort for the person who uses them, and then gradually decrease their efficiency and become substantially useless.

Moreover, the entire surface of the containers reaches a low temperature, and this causes the moisture that is present in the environment to tend to condense on the containers, generating a copious production of water which is an additional discomfort for the person who uses such containers.

### SUMMARY OF THE INVENTION

The aim of the present invention is to provide a device for providing instant cooling, particularly for application to body parts or items, which allows to lower the temperature of the body part or item when it is placed in contact with such part or item.

Within this aim, an object of the present invention is to provide a device for providing instant cooling which can be used at any time and whose duration is not limited by the duration of a chemical reaction which triggers cooling.

Another object of the present invention is to provide a device for providing instant cooling which can be carried conveniently in one of the user's pockets and can be applied in each instance to the body part to be treated.

Still another object of the present invention is to provide a device for providing instant cooling which is capable of maintaining a temperature which is always constant over time.

This aim and these and other objects, which will become better apparent hereinafter, are achieved by a device for providing instant cooling, particularly for treating parts of the human body or items in general, characterized in that it comprises diffuser means which are adapted to be charged with a gas, said diffuser means being provided with a plurality of holes for releasing said gas, said diffuser means being accommodated in a container which contains a thermally insulating material which is adapted to absorb the low temperature generated by said gas, said container being sealed around said diffuser means.

### BRIEF DESCRIPTION OF THE DRAWINGS

Further characteristics and advantages of the invention will become better apparent from the description of a preferred but not exclusive embodiment of the device according to the invention, illustrated by way of non-limiting example in the accompanying drawings, wherein:

FIG. 1 is an exploded perspective view of the device according to the present invention;

FIG. 2 is a perspective view of the propellant bottle to be used in combination with the device according to the present invention;

FIG. 3 is a schematic view of the device according to the invention when used as a bottle-carrying basket.

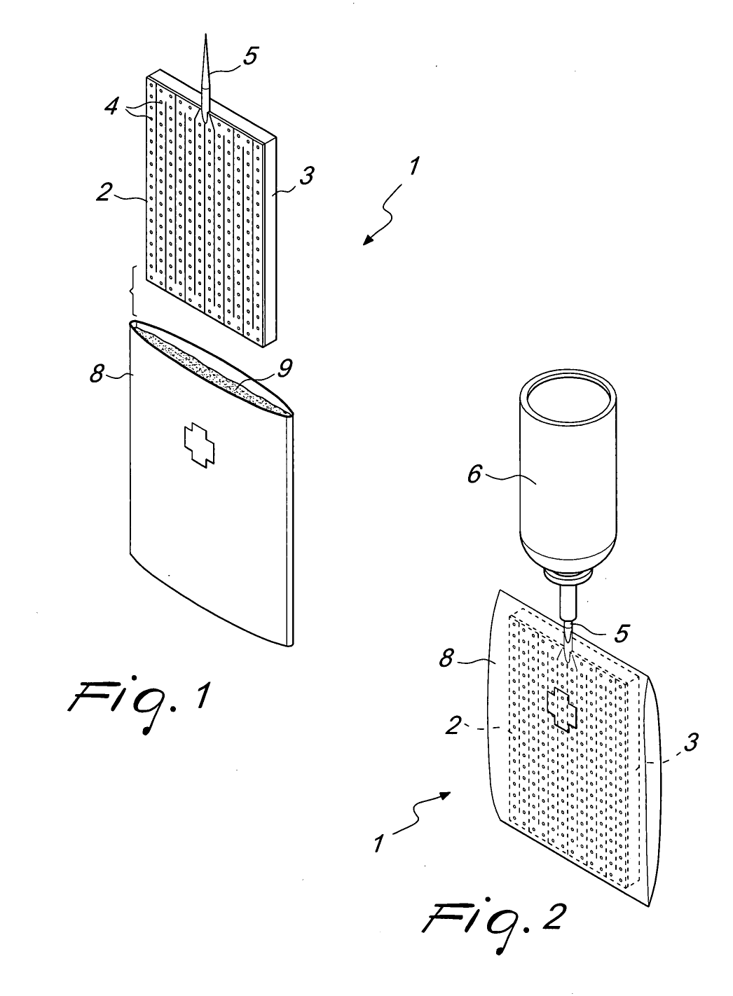

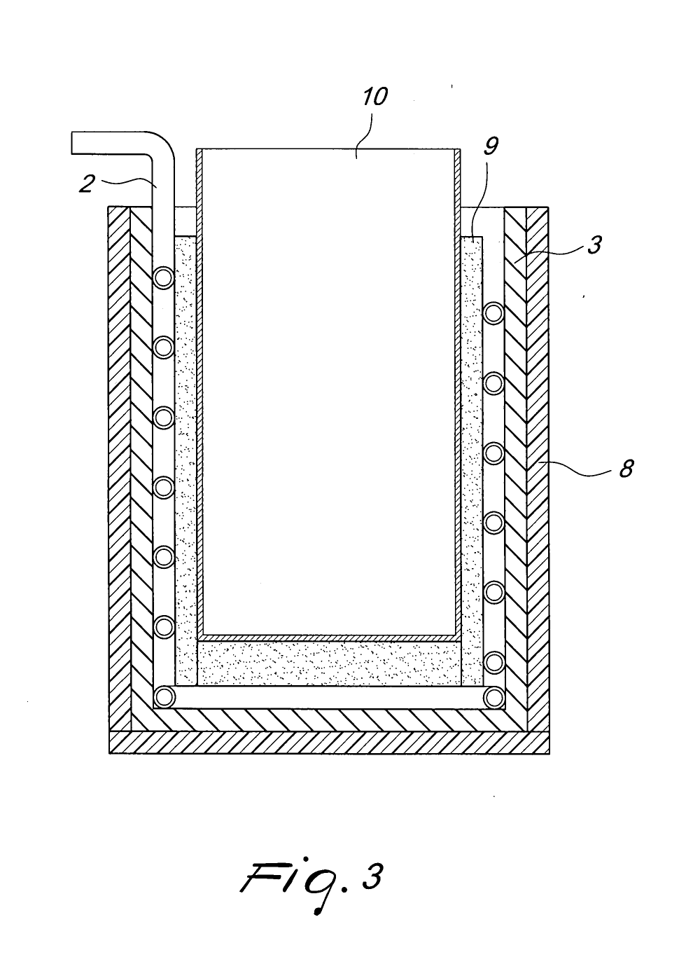

### DESCRIPTION OF THE PREFERRED EMBODIMENTS

With reference to the figures, the device according to the invention, generally designated by the reference numeral 1, comprises means for distributing a gas, which are constituted conveniently by a diffuser 2, which is made for example of sheet metal and is adapted to contain a gas which allows cooling. Conveniently, the diffuser 2 has, at its face designed to be gripped by the user's hand, a layer 3 of thermally insulating material so as to eliminate the discomfort that the user may experience when holding an extremely cold body.

The diffuser 2 has, at the surface that lies opposite the one on which the layer 3 of material is provided, a plurality of holes 4 which are adapted to allow the escape of the gas introduced in the diffuser 2.

Conveniently, the diffuser 2 is provided with means adapted to allow the introduction of the gas in the diffuser, provided for example by a nozzle 5 which allows to connect the diffuser 2 to a bottle 6 of gas, which when needed is connected to the nozzle 5 in order to inject gas into the diffuser 2, and therefore allows the escape of the gas through the holes 4.

Conveniently, the diffuser 2 is accommodated within a containment pouch or container 8, which contains a thermally insulating material, designated by the reference numeral 9, which is constituted for example by soapstone powder with a suitable particle size, which has the task of absorbing the low temperature generated by the expanding gas and of retaining it as long as possible as a function of the amount of soapstone powder and of the amount of gas.

The pouch 8 is sealed around the diffuser 2, with only the nozzle 5 protruding from the pouch 8, in order to allow the connection of the bottle 6 of propellant.

Operation of the device according to the present invention is as follows.

If it is necessary to have instant cold, the user connects the bottle 6 of propellant to the nozzle 5 of the diffuser 2, which is accommodated within the pouch 8, and the gas immediately begins to expand from the diffuser 2 through the holes 4 into the pouch 8, and the soapstone powder absorbs the low temperature that is thus generated.

The soapstone powder is designed to maintain as long as possible the selected low temperature, which can also be a function of the amount of gas injected into the diffuser 2. Therefore, depending on the application, it is possible to give the user an indication as to how much gas he will have to introduce in the diffuser 2 in order to obtain a given temperature.

The device according to the invention is perfectly reusable, since it is only necessary optionally to replace the bottle 6 of propellant in order to have a fresh fill of gas.

The device according to the invention is particularly compact in its dimensions, can be carried easily and can be applied at any time to any part of the body, or to any item, without the generated cold temperature undergoing rapid decay over time and most of all without the device, once in contact with the environment, forming condensation and generating a copious production of water, as occurs with known types of devices.

Conveniently, the diffuser 2 is made of very thin sheet metal, so as to allow it to be highly flexible, indeed to allow the user to adapt the diffuser 2, accommodated in the pouch 8, in the best possible manner to the affected body part.

The device according to the invention, with the same structure described above, can also be used for applications which are different from the one proposed above.

In particular, the device can be provided with an outer container or pouch 8 shaped like a basket and therefore can act as a bottle-carrying basket, so as to allow to preserve the bottles at a given temperature.

In this case, the container can be made of plexiglass or other material, with the layer 3 of thermally insulating material arranged internally to the container. The diffuser means 2 are then arranged, with the thermally insulating means 9 inside the container 8, leaving a free space at the center for accommodating a bottle-container 10.

Another possible application of the device according to the invention provides for its integration within a protective helmet, such as a motorcyclist's helmet and the like. In this case, the purpose would be to keep at a controlled temperature the head of the person who wears the helmet, preventing, especially in the summer months, the heat within the helmet from becoming excessive.

Further applications can provide for use for example in rucksacks, picnic bags and the like.

The device thus conceived is susceptible of numerous modifications and variations, all of which are within the scope of the appended claims; all the details may further be replaced with other technically equivalent elements.

In practice, the materials used, as well as the contingent shapes and dimensions, may be any according to requirements and to the state of the art.

The disclosures in Italian Patent Application no. MI2006A002278, from which this application claims priority, are incorporated herein by reference.

What is claimed is:

1. A device for providing instant cooling, particularly for treating parts of the human body or items in general, comprising diffuser means which are adapted to be charged with a gas, said diffuser means being provided with a plurality of holes for releasing said gas, said diffuser means being accommodated in a container which contains a thermally insulating material which is adapted to absorb low temperature generated by said gas, said container being sealed around said diffuser means.

2. The device according to claim 1, wherein said diffuser means are provided, at one of their surfaces, with a layer of thermally insulating material, which is arranged opposite a surface of said diffuser means in which said plurality of holes is defined.

3. The device according to claim 1, wherein said diffuser means are made of flexible sheet metal.

4. The device according to claim 1, wherein said diffuser means are provided with at least one nozzle for injecting gas into said diffuser means.

5. The device according to claim 1, wherein said thermally insulating material introduced in said container is adapted to freeze upon contact with said gas.

6. The device according to claim 1, wherein said container is made of fabric.

7. The device according to claim 1, wherein said container defines a central space to accommodate a bottle container, said bottle container being surrounded by said thermally insulating material and said diffuser means.

8. A kit for providing instant cooling, particularly for treating body parts or items in general, comprising a device according to claim 1 and a bottle of propellant which is adapted to inject gas into said diffuser means.
